(19) United States
(12) Patent Application Publication
(10) Pub. No.: US 2011/0092842 A1
(43) Pub. Date: Apr. 21, 2011

(54) IMPLANTABLE NEURAL SIGNAL ACQUISTION APPARATUS

(75) Inventors: Christine Decaria, Salt Lake City, UT (US); Michael Sorenson, Salt Lake City, UT (US)

(73) Assignee: I2S MICRO IMPLANTABLE SYSTEMS, LLC, Salt Lake City, UT (US)

(21) Appl. No.: 12/906,792

(22) Filed: Oct. 18, 2010

Related U.S. Application Data
(60) Provisional application No. 61/252,698, filed on Oct. 18, 2009.

(51) Int. Cl. A61B 5/04

(52) U.S. Cl. 600/544

(57) ABSTRACT

In an embodiment, an implantable neural signal acquisition apparatus includes a plurality of electrodes, an implantable electronics package, and a wire bundle. The electrodes are configured to be subcutaneously implanted within neural tissue of a test subject and to collect analog neural signals from the test subject. The implantable electronics package is configured to be subcutaneously implanted within the test subject and to convert the analog neural signals to digital output. The wire bundle is coupled between the electrode array and the implantable electronics package and is configured to convey the analog neural signals from the electrodes to the implantable electronics package.

### CROSS-REFERENCE TO RELATED APPLICATIONS

This application claims the benefit of U.S. Provisional Application No. 61/252,698 filed on 18 Oct. 2009, the disclosure of which is incorporated herein, in its entirety, by this reference.

### BACKGROUND

Neuralphysiological data acquisition systems are configured to record and analyze animal or human brain and/or peripheral-nerve electrical activity. Such systems typically include one or more sensors that generate neural signals indicative of the brain or peripheral-nerve electrical activity near the sensor. Neural signals generated by the sensors can be collected and processed to assist in the study of, for example, sensory perception, motor control, learning and memory, attention, cognition and decision making, drug and toxin effects, epilepsy, Parkinson's, neuroprosthetics, brain-machine interfaces, neurostimulation therapies, dystonia, traumatic brain injury, and stroke.

Some sensors are implanted subcutaneously within a test subject and each sensor is typically connected to a separate wire that connects the sensor within the test subject to a system external to the test subject. Several wires may be bundled together in a cable, sometimes called a pigtail, and each pigtail exits the test subject's skin at a separate incision site. Some pigtails include, for example, 16 wires connected to 16 different sensors. For sensor arrays having a large number of sensors, such as 128 sensors, at least 8 different pigtails may be provided that exit the test subject's skin at 8 different incision sites. More sensors typically require more pigtails and more incision sites, increasing the risk for infection and the number of scars for the test subject. Thus, it is desirable to implant as little hardware as possible within a test subject for the collection of neural signals.

The neural signals generated by the sensors are analog neural signals typically having a voltage on the order of hundreds of microvolts (“μV”) and the wires connected to the sensors are relatively high-impedance wires having impedances of about 100-800 kilo-ohms. The combination of low voltage and high impedance prevents the analog neural signals from being accurately transmitted far from the signal source.

Accordingly, a front-end amplifier is often provided external to the test subject to receive and condition the analog neural signals before providing the conditioned signals to an external processing system. The front-end amplifier may, among other things, amplify the analog neural signals for subsequent processing by an external processing system.

Therefore, what are needed are improved methods and systems for conditioning neural signals collected by neural sensors.

### SUMMARY

Some embodiments generally relate to an implantable neural signal acquisition apparatus configured to condition analog neural signals within a test subject.

In an embodiment, an implantable neural signal acquisition apparatus includes a plurality of electrodes, an implantable electronics package, and a wire bundle. The electrodes are configured to be subcutaneously implanted within neural tissue of a test subject and to collect analog neural signals from the test subject. The implantable electronics package is configured to be subcutaneously implanted within the test subject and to convert the analog neural signals to digital output. The wire bundle is coupled between the electrode array and the implantable electronics package and is configured to convey the analog neural signals from the electrodes to the implantable electronics package.

In an embodiment, a neuralphysiological data acquisition system includes an implantable neural signal acquisition apparatus and an external neural signal processor. The implantable neural signal acquisition apparatus includes an electrode array, a wire bundle, and an implantable electronics package. The electrode array is configured to be subcutaneously implanted within neural tissue of a test subject. The wire bundle is coupled to the electrode array and is configured to be subcutaneously implanted within the test subject. The implantable electronics package is coupled to the wire bundle and is configured to be subcutaneously implanted within the test subject. The implantable electronics package is further configured to convert the analog neural signals to digital output. The external neural signal processor is coupled to the implantable neural signal acquisition apparatus.

In an embodiment, a method of collecting and conditioning neural signals includes collecting analog neural signals from neural tissue of a test subject. The method additionally includes conditioning the collected analog neural signals within the test subject to generate a single digital output representing the collected analog neural signals. The method additionally includes transmitting the single digital output outside of the test subject to an external processing system.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical elements or features in different views or embodiments shown in the drawings.

FIG. 1 illustrates an example operating environment in which embodiments of a neuralphysiological data acquisition system including an implantable neural signal acquisition apparatus can be implemented;

FIGS. 2A and 2B are functional block diagrams of two different embodiments of the neuralphysiological data acquisition system of FIG. 1;

FIG. 3A illustrates an embodiment of the implantable neural signal acquisition apparatus of FIG. 1;

FIG. 3B illustrates an example implementation of the implantable neural signal acquisition apparatus of FIG. 3B implanted in a test subject;

FIGS. 4A and 4B are cross-sectional views of two implantable electronics package embodiments such as may be included in the implantable neural signal acquisition apparatus of FIG. 1;

FIG. 5 is a functional block diagram of an embodiment of an implantable electronics package such as may be included in the implantable neural signal acquisition apparatus of FIG. 1;

FIG. 6 is a functional block diagram of an example amplifier circuit such as may be included in an implantable electronics package according to some embodiments;

FIG. 7A shows a flow diagram of a method of collecting and conditioning analog neural signals within a test subject according to an embodiment; and

FIG. 7B shows a flow diagram of a method of conditioning collected analog neural signals according to an embodiment.

### DETAILED DESCRIPTION

Embodiments of the invention relate to an implantable neural signal acquisition apparatus configured to be subcutaneously implanted in a test subject and to condition analog neural signals within the test subject. Systems including an implantable neural signal acquisition apparatus, such as neuralpsyiological data acquisition systems, and methods implemented by an implantable neural signal acquisition apparatus and/or corresponding systems, are also disclosed.

### I. Example Operating Environment

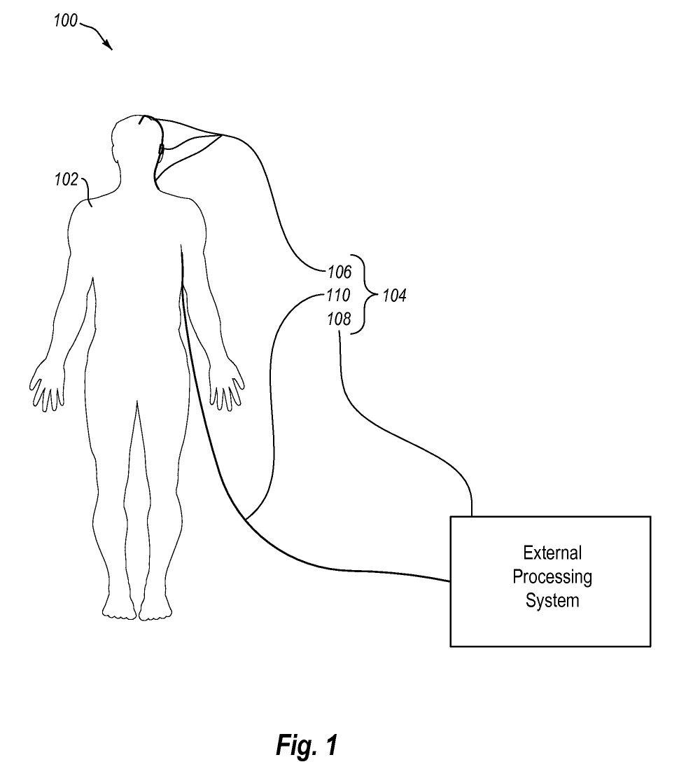

One example operating environment 100 is illustrated in FIG. 1. The example operating environment 100 includes a test subject or patient 102 and a neuralphysiological data acquisition system 104 (hereinafter “system 104”), including an implantable neural signal acquisition apparatus 106 (hereinafter “implantable apparatus 106”) and an external processing system 108. Optionally, the system 104 further includes a transmission channel 110.

In the illustrated embodiment of FIG. 1, the test subject 102 is a human test subject. In other embodiments, the test subject 102 may be an animal test subject, such as an avian, rodent, feline, or primate test subject, or other suitable test subject.

The system 104 is generally configured to collect, record and analyze brain and/or peripheral nerve activity of the test subject 102. Consistent with the foregoing, the implantable apparatus 106 may be configured to collect analog neural signals output by the test subject 102. In addition, the implantable apparatus 106 may be configured to amplify, multiplex and digitize and otherwise condition the collected analog neural signals to generate a digital output for transmission across the communication channel 110. The external processing system 108 is configured to receive and process the digital output for recording and analyzing the brain and/or peripheral nerve activity of the test subject 102. Additional details of the implantable apparatus 106 and system 104 are provided below.

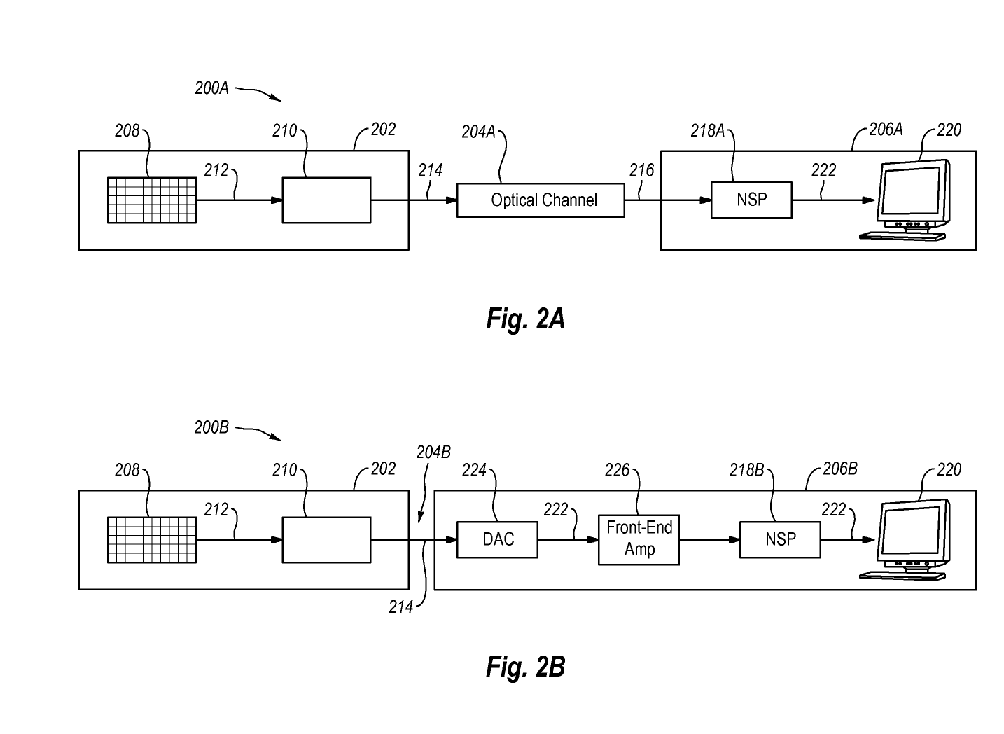

FIG. 2A is a functional block diagram of a first embodiment of a neuralphysiological data acquisition system 200A (hereinafter “system 200A”) such as may be implemented in the example operating environment 100 of FIG. 1. The system 200A may correspond to the system 104 of FIG. 1. In the illustrated embodiment, the system 200A includes an implantable neural signal acquisition apparatus 202 (hereinafter “implantable apparatus 202”), a transmission channel 204A, and an external processing system 206A.

The implantable apparatus 202 may generally include a plurality of electrodes 208 electrically coupled to an implantable electronics package 210. The electrodes 208 are configured in some embodiments to be subcutaneously implanted within neural tissue, such as cortical tissue or peripheral nerve tissue, of a test subject and to collect analog neural signals 212 from the neural tissue of the test subject. Each electrode 208 serves as a neural interface that essentially connects neurons to electronic circuitry. The electrodes 208 may include multiple implantable individual stiff-wire electrodes, an implantable microelectrode or microwire array, planar silicon probes, a subdural electrocorticography (“ECoG”) grid, epidural electroencephalography (“EEG”) electrodes, or other suitable implantable electrodes or electrode arrangement.

Similar to the electrodes 208, the implantable electronics package 210 is also configured in some embodiments to be subcutaneously implanted within a test subject, such as the test subject 102 of FIG. 1. Alternately or additionally, the implantable electronics package 210 is configured to condition the analog neural signals 212 collected by the electrodes 208 to generate a first digital output 214. In more detail, the implantable electronics package 210 may be configured to perform at least one of amplifying the analog neural signals 212, filtering the amplified analog neural signals, multiplexing the filtered analog neural signals to generate multiplexed analog neural signals, digitizing the multiplexed analog neural signals, and/or packetizing the digital neural signals for inclusion in the first digital output 214.

The implantable electronics package 210 is coupled to the transmission channel 204A. In the illustrated embodiment of FIG. 2A, the transmission channel 204A is an optical channel. In general, the transmission channel 204A includes an electro-optical (“EO”) converter, an optical transmission medium, and an opto-electrical (“OE”) converter.

The EO converter is configured to convert the first digital output 214 received from the implantable electronics package 210 into an optical signal for transmission through the optical transmission medium. The EO converter may generally include an optical transmitter, examples of which include, but are not limited to, a light emitting diode (“LED”), a directly modulated laser (“DML”), such as a directly modulated fabry perot (“FP”) laser, distributed feedback (“DFB”) laser, distributed Bragg reflector (“DBR”) laser, vertical cavity surface emitting laser (“VCSEL”), or the like, or an externally modulated laser (“EML”), such as a lithium niobate (“LiNbO₃”) EML, an electroabsorption (“EA”) modulated laser, a Mach Zhender (“MZ”) EML, or the like.

The optical transmission medium may include, for example, an optical fiber or other suitable optical waveguide.

The OE converter is configured to receive the optical signal transmitted through the optical transmission medium and convert it to a second digital output 216. In some examples, the second digital output 216 is in the same format as the first digital output 214. The OE converter may generally include an optical receiver, examples of which include, but are not limited to, a positive-intrinsic-negative (“PIN”) photodiode, or other suitable optical receiver.

According to some embodiments, the inclusion of an optical transmission channel 204A in the system 200A serves to electrically isolate a test subject from earth ground or other potentially hazardous electrical sources to which the external processing system 206A may be electrically coupled. Alternately or additionally, the optical transmission channel 204A permits neural data to be accurately transmitted relatively longer distances than may otherwise be possible over an electrical transmission channel.

The external processing system 206A may include a neural signal processor (“NSP”) 218A coupled to the transmission channel 214 and a general purpose or special purpose computer 220. The NSP 218A is generally configured to perform specific types of signal processing on the second digital output 216 received from the transmission channel 204A and to output one or more processed neural signals 222. For instance, the NSP 218A may be configured to digitally filter the second digital output 216 based on specific filter criteria, or to detect neuron action potentials embedded in the digital output 216, or to use a selected one of the electrodes 208 as a reference electrode for a selected one or more of the other electrodes 208, or to use bipolar differential reporting (i.e., taking the difference between pairs of the electrodes 208), or to filter electrical line noise as described in U.S. patent application Ser. No. 12/906,673 entitled METHODS AND SYSTEMS FOR SIGNAL PROCESSING OF NEURAL DATA, filed Oct. 18, 2010, the disclosure of which is incorporated herein, in its entirety, by this reference.

Optionally, the NSP 218A can be coupled directly to the implantable electronics package 210 via one or more electrical wires to directly receive first digital output 214 in which case the optical transmission channel 204A can be omitted.

The computer 220 is a desktop or laptop computer or other computing device coupled to the NSP 218A. The computer 220 may be configured to receive the processed neural signals 222, display the processed neural signals 222, or perform certain functions on the processed neural signals 222.

FIG. 2B is a functional block diagram of a second embodiment of a neuralphysiological data acquisition system 200B (hereinafter “system 200B”) such as may be implemented in the example operating environment 100 of FIG. 1. The system 200B may correspond to the system 104 of FIG. 1. The system 200B is similar in some respects to the system 200A of FIG. 2B and like references numbers are used to designate like components.

Various differences between the system 200B of FIG. 2B and the system 200A of FIG. 2A will now be explained. First, the system 200B of FIG. 2B includes a transmission channel 204B that generically represents any suitable transmission channel. For instance, the transmission channel 204B may include a wireless RF transmission channel, a hardwired transmission channel, an optical transmission channel, or other suitable transmission channel.

Additionally, in the example of FIG. 2A, the NSP 218A is configured to process digital signals, such as the second digital output 216, in a first digital format. While the NSP 218B of FIG. 2B is also configured to process digital signals, the NSP 218B of FIG. 2B is configured to process digital signals in a second digital format different than the first digital format.

To this end, the external processing system 206B includes a digital-to-analog converter (“DAC”) 224 configured to convert the first digital output 214 in the first digital format received from the implantable electronics package 210 to an analog output 225.

Additionally, a front-end amplifier 226 is coupled between the DAC 224 and the NSP 218B. The front-end amplifier 226 is configured to, among other things, convert the analog output 225 to a digital output 228 in a second digital format different than the first digital format.

The NSP 218B receives the digital output 228 of the front-end amplifier 226 in the second digital format and performs one or more signal processing functions on the digital output 228, such as one or more of the signal processing functions described above with respect to the NSP 218A of FIG. 2A.

### II. Example Implantable Apparatus

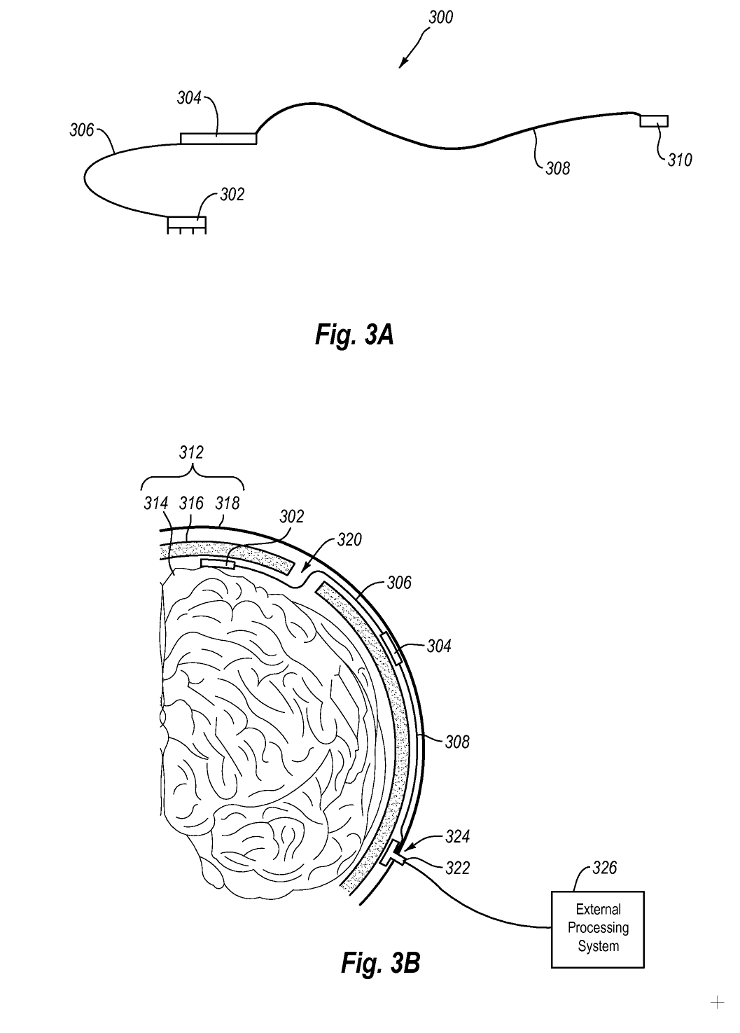

FIG. 3A illustrates an example of an implantable neural signal acquisition apparatus 300 (“implantable apparatus 300”) according to some embodiments. The implantable apparatus 300 is configured to be implemented in environments and/or systems such as illustrated in FIGS. 1-2B. Accordingly, the implantable apparatus 300 may correspond to one or more of the implantable apparatuses 106 or 202 of FIGS. 1-2B.

In the illustrated embodiment of FIG. 3A, the implantable apparatus 300 includes a plurality of electrodes 302 and an implantable electronics package 304. The electrodes 302 and implantable electronics package 304 may correspond to, respectively, the electrodes 208 and implantable electronics package 210 if FIGS. 2A-2B, for instance.

The implantable apparatus 300 additionally includes a first or proximal wire bundle 306 and a second or distal wire bundle 308. The first wire bundle 306 is coupled between the electrodes 302 and the implantable electronics package 304. Generally, the first wire bundle 306 includes at least one wire per electrode. Accordingly, when the electrodes 302 include an array of, for instance, 96, 128, or 256 electrodes, the first wire bundle 306 in some examples includes at least 96, 128, or 256 wires, respectively. Further, the first wire bundle 306 may be configured to convey analog neural signals collected by the electrodes 302 from the electrodes 302 to the implantable electronics package 304.

The first wire bundle 306 may have a length of several centimeters (“cm”). In some embodiments, the length of first wire bundle 306 may be about 13 cm. In other embodiments, the length of first wire bundle 306 may range from about 1.5 cm to about 30 cm, or from about 5 cm to about 24 cm.

The second wire bundle 308 is coupled to an output of the implantable electronics package 304. The second wire bundle 308 is configured to convey a digital output of the implantable electronics package 304 to an external processing system, such as the external processing system 108, 206A, 206B of FIGS. 1-2B.

In some embodiments, the second wire bundle 308 includes seven distinct wires, including thee wires for power (e.g., ground, positive supply voltage and negative supply voltage), two wires for a differential input clock, and two wires for differential data output. Alternately or additionally, the second wire bundle 308 is a single pigtail-type cable having a plurality of ring contacts on a distal end of the second wire bundle 308.

Optionally, the implantable apparatus 300 further includes a connector 310 attached to a distal end of the second wire bundle 308. The connector 310 is configured to provide a mechanical and electrical interface between the implantable system 300 and an external processing system.

As previously indicated herein, embodiments of the implantable apparatuses 106, 202, 300 are configured to be implanted subcutaneously in a test subject. In this regard, FIG. 3B illustrates an example implementation in which the implantable apparatus 300 of FIG. 3A is implanted in a test subject or patient 312.

As shown, the test subject 312 includes cortical tissue 314, cranium 316, and skin 318. A hole 320 drilled in the cranium 316 permits the electrodes 302 to be implanted subcutaneously within the cortical tissue 314. More particularly, the electrodes 302 are implanted subcranially in FIG. 3B. Although the electrodes 302 are shown as being disposed on the surface of the cortical tissue 314 between the cranium 316 and the cortical tissue 314, the electrodes 302 may alternately or additionally penetrate into the cortical tissue 314.

The implantable electronics package 304 is also implanted subcutaneously. In particular, the implantable electronics package 304 is implanted between the skin 318 and the cranium 316.

The first wire bundle 306 electrically couples the electrodes 302 to the implantable electronics package 304 through the hole 320 formed in the cranium 316.

The second wire bundle 308 is coupled to an output of the implantable electronics package 304 and may terminate subcutaneously at a pedestal 322 fixedly positioned at an incision site 324, the pedestal 322 providing an interface to an external processing system 326. The pedestal 322 may be included as part of the implantable system 300 in some embodiments.

Alternately, the second wire bundle 308 may extend through the incision site 324 to terminate outside of the test subject, where the distal end of the second wire bundle 308 can be connected through an appropriate interface to the external processing system 326.

According to some embodiments, the implantability of implantable electronics package 304 and other implantable electronics packages described herein permits the implantable electronics package 304 to be located proximate to the electrodes 302 where amplification and other conditioning functions can be performed on collected analog neural signals prior to transmitting the conditioned neural signals outside of the test subject 312. The proximity of the implantable electronics package 304 to the electrodes 302 and the amplification and other functions performed by the implantable electronics package 304 substantially reduce or eliminate noise otherwise introduced in some systems in which un-amplified analog neural signals are transmitted outside the test subject to an external front-end amplifier. In particular, movement artifacts, electrical line noise and signal degradation can be substantially reduced or eliminated by reducing the length of the high-impedance wires over which the un-amplified analog neural signals are transmitted by implanting the implantable electronics package within the test subject 312 near the electrodes 302, and by conditioning the analog neural signals prior to transmission outside the test subject 312.

Thus, despite conventional wisdom teaching that the amount of hardware implanted within a test subject for collecting neural signals should be minimized, the present application nevertheless appreciates that by moving conditioning functions (e.g., provided by implantable electronics package 304) inside the test subject, movement artifacts, electrical line noise, and signal degradation can be reduced. Moreover, as will be described in greater detail below, the configuration of some embodiments of the implantable electronics packages disclosed herein ultimately reduces the amount of hardware implanted in the test subject compared to some conventional systems, by, e.g., multiplexing collected neural signals to reduce the number of wires required to transmit neural signal data outside of the test subject.

### III. Example Implantable Electronics Package

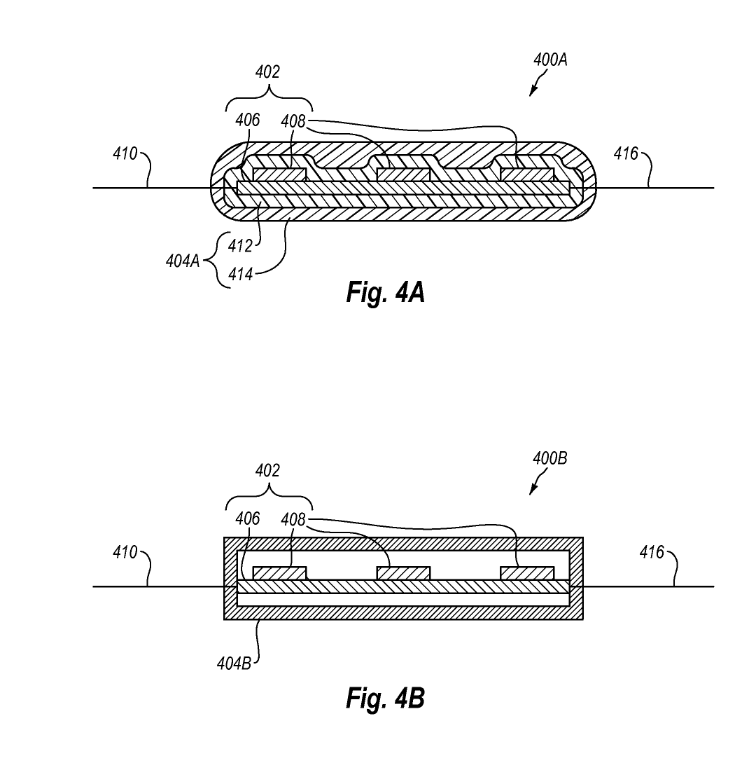

FIG. 4A illustrates a cross-sectional view of an implantable electronics package 400A according to some embodiments. The implantable electronics package 400A may correspond to one or more of the implantable electronics packages 210, 304 of FIGS. 2A-3B, for example.

In the illustrated embodiment of FIG. 4A, the implantable electronics package 400A includes a printed circuit board assembly (“PCBA”) 402 and a bio-compatible housing 404A. More generally, the implantable electronics package 400A may include one or more electronics encapsulated within bio-compatible housing 404A.

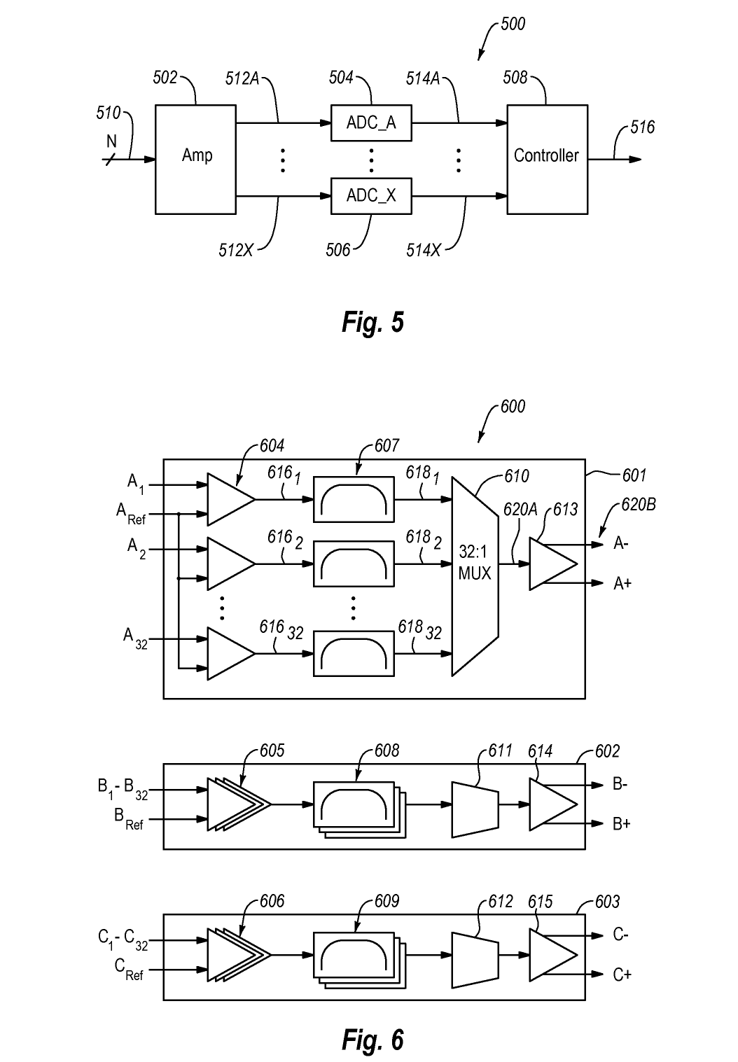

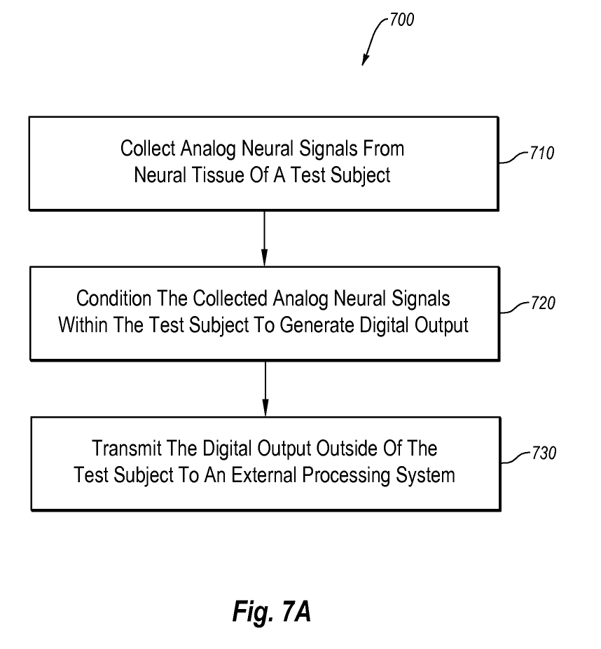

The PCBA 402 may include a printed circuit board (“PCB”) 406 and a plurality of integrated circuits (“ICs”) 408 attached to the PCB 406. In some embodiments, the ICs 408 include, for example, an amplifier IC, one or more analog-to-digital converter (“ADCs”) ICs, and a controller IC, aspects of which are explained in greater detail below with respect to FIGS. 5-6.

The bio-compatible housing 404A is configured to encapsulate the PCBA 402 and generally prevent direct interaction between the ICs 408 or other components of the PCBA 402 with surrounding tissue of a test subject. Accordingly, the bio-compatible housing 404A may include one or more biomaterials, e.g., natural or synthetic material(s) that is (are) suitable for introduction into living tissue. For example, the bio-compatible housing 404A in some embodiments includes a polymer layer 412 substantially coating the PCBA 402 and a bio-compatible silicone layer 414 coating the polymer layer 412.

The polymer layer 412 may include Parylene or other suitable polymer. In general, Parylene includes derivatives of p-xylylene, such as, but not limited to, di-p-xylylene (also known as paracyclophane), Parylene N (hydrocarbon), Parylene C (one chlorine group per repeat unit), Parylene D (two chlorine groups per repeat unit), Parylene AF-4 (aliphatic fluorination 4 atoms), Parylene SF, Parylene HT, Parylene A (one amine per repeat unit), Parylene AM (one methylene amine group per repeat unit), Parylene VT-4 (fluorine atoms on the aromatic ring), or other suitable p-xylylene derivative.

The polymer layer 412 is generally configured to function as a moisture barrier and/or electrical insulator between the PCBA 402 and the bio-compatible silicone layer 414 and/or surrounding tissue of a test subject. Accordingly, any suitable polymer material, including, but not limited to, Parylene, Para-xylene, polyimide, polyurethane, epoxy, or the like, can be implemented in the polymer layer 412. Alternately or additionally, any non-polymer material—such as, but not limited to, Silicon Nitride—having the appropriate characteristics to function as a moisture barrier and/or electrical insulator can be substituted for the polymer layer 412.

The layer 414 is generally configured for introduction into living tissue without causing any serious adverse affects, such as rejection by the body of the test subject. While the layer 414 has been described as including bio-compatible silicone, any other suitable material(s) can be implemented in the layer 414. Examples of other suitable materials that can be used in the layer 414 include, but are not limited to, polymers, including Para-xylene, polyimide, polyurethane, epoxy, or the like.

The first wire bundle 410 and a second wire bundle 416 are configured to penetrate through the bio-compatible housing 404A and couple to the PCBA 406.

The implantable electronics package 400A of FIG. 4A in some embodiments is configured for use in acute settings involving implantation of the implantable electronics package 400A within a test subject for, e.g., 30 days or less.

FIG. 4B illustrates a simple cross-sectional view of an example of another implantable electronics package 400B according to some embodiments. The implantable electronics package 400B may correspond to one or more of the implantable electronics packages 210, 304 of FIGS. 2A-3B, for example. The implantable electronics package 400B is similar in some respects to the implantable electronics package 400A of FIG. 4A and like reference numbers are used to designate like components.

In contrast to the implantable electronics package 400A of FIG. 4A, the implantable electronics package 400B of FIG. 4B includes a different bio-compatible housing 404B. In particular, the bio-compatible housing 404B may include titanium, a titanium alloy, stainless steel, other suitable metal(s), ceramic(s), or any combination thereof. Additionally, feed-throughs (not shown) may be provided in the bio-compatible housing 404B through which the first and second wire bundles 410, 415 electrically couple to the PCBA 402 encapsulated by bio-compatible housing 404B. The implantable electronics package 400B of FIG. 4B in some embodiments is configured for use in chronic settings involving implantation of the implantable electronics package 400B within a test subject for more than, e.g., 30 days.

FIG. 5 is a functional block diagram of an implantable electronics package 500 that may correspond to one or more of the implantable electronics packages 210, 304, 400A, 400B of FIGS. 2A-4B, for instance. The implantable electronics package 500 includes a plurality of circuits 502, 504, 506, 508, including an amplifier circuit 502, a plurality of ADC circuits 504, 506, and a controller circuit 508. The circuits 502, 504, 506, 508 may correspond to the ICs 408 of FIGS. 4A-4B, for example.

The amplifier circuit 502 is configured to receive a plurality, e.g., N, of analog neural signals 510 from a plurality N of electrodes (not shown). The amplifier circuit 502 is further configured to, among other things, amplify the analog neural signals and multiplex the amplified analog neural signals into a plurality, e.g., X, of multiplexed analog neural signals 512A-512X (collectively “multiplexed analog neural signals 512”), where X is less than N.

In some embodiments, N is 96 and X is 3. In other embodiments, N may be virtually any number such as, but not limited to, 128 or 256. Similarly, X may be virtually any number such as, but not limited to, 4. Optionally, the amplifier circuit 502 may additionally perform filtering on the amplified analog neural signals.

The ADC circuits 504, 506 are each coupled to a respective output of the amplifier circuit 502. Each of ADC circuits 504, 506 is configured to receive a separate one of the X multiplexed analog neural signals 512 and to convert the corresponding multiplexed analog neural signal 512 from an analog signal to a digital signal 514A-514X (collectively “digital signals 514”).

The controller circuit 508 is coupled to respective outputs of the ADCs 504, 506. The controller circuit 508 may be configured to control operation of the amplifier circuit 502 and ADC circuits 504, 506. Alternately or additionally, the controller circuit 508 is configured to receive each digital signal 514 output by the ADC circuits 504, 506 and to packetize the received digital signals 514 to generate a single digital output 516. The digital output 516 is then provided from the implantable electronics package 500 to an external processing system.

Although not shown in FIG. 5, the implantable electronics package 500 may further include a low-voltage differential signaling (“LVDS”) circuit coupled to the output of the controller circuit 508 and configured to transmit the digital output 516 to the external processing system.

Alternately or additionally, the implantable electronics package 500 of FIG. 5 may further include an a/c coupled protection circuit (not shown) configured to prevent electrical signals from traveling to the electrodes connected to the input of the amplifier circuit 502 and/or to the test subject.

FIG. 6 is a functional block diagram of an amplifier circuit 600 that may correspond to, for example, the amplifier circuit 502 of FIG. 5, for instance. The amplifier circuit 600 includes a plurality of circuit elements arranged in a plurality of conditioning banks 601, 602, 603. Each conditioning bank 601-603 may be configured to condition a respective group of incoming analog neural signals A₁-A₃₂, B₁-B₃₂, or C₁-C₃₂ collected by a respective bank of electrodes (not shown) connected to an input of each conditioning bank 601-603. In the illustrated embodiment, for example, the amplifier circuit 600 includes three conditioning banks 601-603 configured to condition incoming analog neural signals A₁-A₃₂, B₁-B₃₂, or C₁-C₃₂ collected by three corresponding banks of electrodes including 32 electrodes each.

With combined reference to FIGS. 5 and 6, the number X of ADC circuits 504, 506 in an implantable electronics package 500 in some embodiments is the same as the number of conditioning banks 601-603 of the amplifier circuit 502 implemented in the implantable electronics package 500. Accordingly, for an implantable electronics package 500 including the amplifier circuit 600 with three conditioning banks 601-603, the implantable electronics package 500 may include three ADC circuits, represented by ADC circuits ADC_A (504) through ADC_X (506) in FIG. 5.

Returning to FIG. 6, each conditioning bank 601-603 may include a plurality of pre-amplifiers 604-606, a plurality of filters 607-609, and a respective multiplexer 610-612. Optionally, each conditioning bank 601-603 further includes a respective differential driver 613-615.

Generally, each of the conditioning banks 601-603 operates and is configured in a similar manner. For example, despite the differences in the depictions in FIG. 6 of conditioning banks 602 and 603 with respect to conditioning bank 601, conditioning banks 602 and 603 are generally configured and operate in a similar manner as conditioning bank 601. Consistent with the foregoing, aspects of the configuration and operation of conditioning bank 601 will now be described, with the understanding that conditioning banks 602, 603 may be configured and operated in a similar manner.

In some embodiments, the conditioning bank 601 is configured to receive, from a corresponding electrode bank, a plurality of analog neural signals A₁, A₂, . . . A₃₂ (collectively “analog neural signals A₁-A₃₂”). In particular, in the illustrated embodiment, the conditioning bank 601 receives 32 analog neural signals A₁-A₃₂, while in other embodiments the conditioning bank 601 receives more or less than 32 analog neural signals A₁-A-₃₂.

Optionally, the conditioning bank 601 is additionally configured to receive a reference signal A_{Ref} with respect to which the analog neural signals A₁-A₃₂ may be differentially amplified. The reference signal A_{Ref} may be received from, e.g., a reference electrode, such as a platinum wire, implanted in the head. In some embodiments, the reference signals A_{Ref}, B_{Ref}, C_{Ref} received by each conditioning bank 601-603 are collected by the same reference electrode, while in other embodiments two or more of the reference signals A_{Ref}, B_{Ref}, C_{Ref} are collected by different reference electrodes.

The pre-amplifiers 604 of conditioning bank 601 generally include one pre-amplifier 604 for each input signal A₁-A₃₂. Alternately or additionally, the filters 607 of conditioning bank 601 include one filter 607 for each input signal A₁-A₃₂. Accordingly, in the illustrated embodiment, the pre-amplifiers 604 include 32 pre-amplifiers 604 and the filters 607 include 32 filters 607, while in other embodiments the pre-amplifiers 604 and filters 607 may include more or less than 32 pre-amplifiers 604 or 32 filters 607.

The pre-amplifiers 604 have a 100× gain in some embodiments. In other embodiments, a gain of each of pre-amplifiers 604 is more or less than 100×. Further, the pre-amplifiers 604 in some embodiments are configured to differentially amplify the analog neural signals A₁-A₃₂ with respect to the reference signal A_{Ref}.

The filters 607 are bandpass filters in some embodiments. The passband of the filters 607 may be configured such that a high-frequency stop-band of the filters 607 substantially eliminates or reduces aliasing caused by high frequency noise and a low-frequency stop-band of the filters 607 substantially eliminates or reduces a DC offset that might otherwise saturate the amplifier circuit 600.

The multiplexer 610 in the illustrated embodiment is a 32:1 multiplexer. More generally, the multiplexer 610 may be an (N/X):1 multiplexer, where N is the total number of analog neural signals—excluding reference signals A_{Ref}, B_{Ref}, C_{Ref}—received by the amplifier circuit 600 (e.g., N=96 in FIG. 6) and X is the number of conditioning banks 601, 602, 603 (e.g., X=3 in FIG. 6) included in the amplifier circuit 600.

In operation, the pre-amplifiers 604 receive and amplify respective ones of the analog neural signals A₁-A₃₂ to generate amplified analog neural signals 616₁, 616₂, . . . 616₃₂ (collectively “amplified analog neural signals 616₁-616₃₂”).

The amplified analog neural signals 616₁-616₃₂ are selectively filtered by filters 607 to remove unwanted frequencies from the amplified analog neural signals 616₁-616₃₂. Filtering the amplified analog neural signals 616₁-616₃₂ generates filtered analog neural signals 618₁, 618₂, . . . 618₃₂ (collectively “filtered analog neural signals 618₁-618₃₂”).

The filtered analog neural signals 618₁-618₃₂ are multiplexed into a serial single-ended signal 620A by multiplexer 610. The single-ended signal 620A can be provided directly to a corresponding ADC circuit, such the ADC circuits 504, 506 of FIG. 5, or may be converted to a serial differential signal pair 620B as shown in FIG. 6 by differential driver 613 before being provided to the corresponding ADC circuit. The single-ended signal 620A or differential signal pair 620B ultimately provided to the corresponding ADC circuit may be generically referred to herein as “multiplexed analog neural signal 620.”

As described above with respect to FIG. 5, the multiplexed analog neural signals output by conditioning banks 601-603 may be digitized by corresponding ADC circuits and packetized by a control circuit into a single digital output.

According to some embodiments, multiplexing the filtered analog neural signals (including filtered analog neural signals 618₁-618₃₂) derived from analog neural signals A₁-A₃₂, B₁-B₃₂ and C₁-C₃₂ ultimately serves to reduce the number of wires that are required to convey data representing the analog neural signals A₁-A₃₂, B₁-B₃₂ and C ₁-C₃₂ outside of a test subject. In particular, some systems including, for instance, a 16×8 array of electrodes, require one pigtail for each set of 16 electrodes. In other words, a 16×8 array of electrodes may include 8 pigtails to convey the analog neural signals collected by the 16×8 array of electrodes outside of a test subject. Typically, each pigtail exits the test subject's skin through a separate incision such that a 16×8 array of electrodes with 8 pigtails implemented in a test subject will require 8 separate incisions for the 8 pigtails.

In contrast, some embodiments disclosed herein multiplex the collected analog neural signals to a relatively small number of multiplexed analog neural signals as has already been described herein. After digitization, the corresponding digital signals are also packetized into a single digital output, which may be conveyed outside the test subject over a differential signal pair requiring a mere two wires. Although several additional wires may be coupled to the implantable electronics package for, e.g., power and clock signals, the total number of wires that exit a test subject according to some embodiments can be reduced to a fraction of the total number of wires connected to the electrodes such that a single pigtail exits the test subject in some embodiments. For instance, in the present example, an implantable electronics package connected to a single pigtail including 7 wires can be used to condition 96 analog neural signals and convey data representing the 96 analog neural signals outside of the test subject. Further, a single pigtail requires a single incision, thereby reducing the number of incisions (and resulting scars) and risk of infection in a test subject compared to systems including numerous pigtails.

### IV. Example Methods of Operation

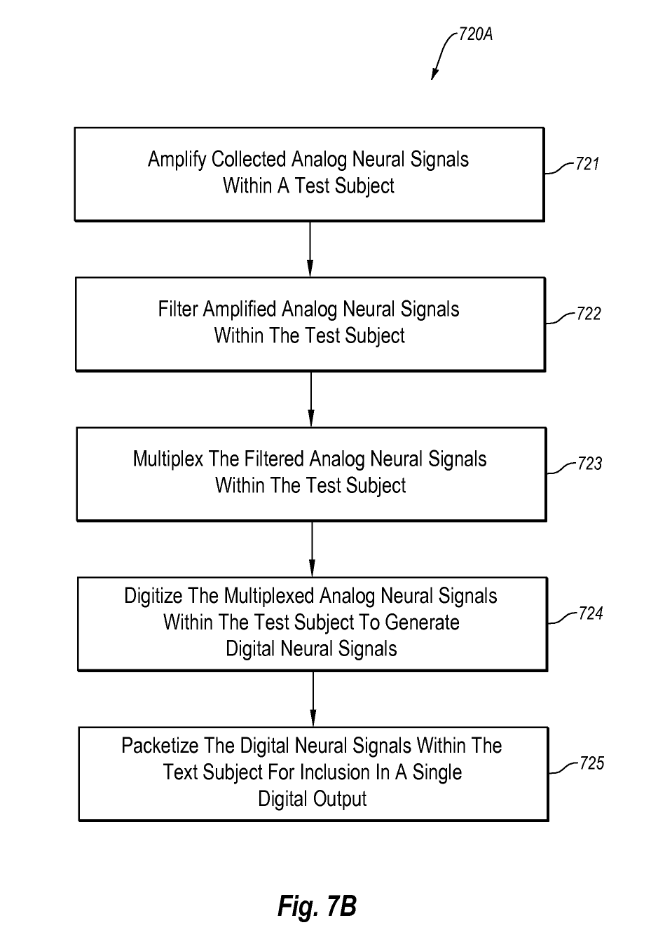

Turning next to FIGS. 7A and 7B, various example methods of operation are described according to some embodiments. One skilled in the art will appreciate that, for processes and methods disclosed herein, the acts performed in the processes and methods may be implemented in differing order than disclosed herein. Furthermore, the outlined acts and operations are only provided as examples, and some of the acts and operations may be optional, combined into fewer acts and operations, or expanded into additional acts and operations without detracting from the essence of the disclosed embodiments.

FIG. 7A is a flowchart of an example method 700 of collecting and conditioning analog neural signals within a test subject. The method 700 of FIG. 7A is implemented in some embodiments by an implantable neural signal acquisition apparatus, such as the implantable apparatuses 106, 202, 300 of FIGS. 1-3B, including a plurality of electrodes, such as the electrodes 208, 302 of FIGS. 2A-3B, and an implantable electronics package, such as the implantable electronics packages 210, 304, 400A, 400B, 500 of FIGS. 2A-5.

The method 700 begins in some embodiments by collecting 710 analog neural signals from neural tissue of a test subject. The act 710 of collecting analog neural signals is performed in some embodiments by electrodes included in an implantable apparatus.

The method 700 additionally includes conditioning 720 the collected analog neural signals within the test subject to generate a single digital output representing the collected analog neural signals. The act 720 of conditioning the collected analog neural signals within the test subject to generate a single digital output is performed in some embodiments by an implantable electronics package included in the implantable apparatus. An example of the conditioning that may be involved in act 720 is disclosed with respect to FIG. 7B.

The method 700 additionally includes transmitting 730 the single digital output outside of the test subject to an external processing system. The act 730 of transmitting the single digital output outside of the test subject to the external processing system may be performed by an LVDS circuit included in the implantable electronics package.

Alternately or additionally, the act 730 of transmitting the single digital output outside of the test subject to the external processing system may include converting the digital output to an optical signal and transmitting the optical signal to the external processing system via an optical transmission channel, such as the transmission channel 204A of FIG. 2A.

Other acts and operations not shown in FIG. 7A or described above can optionally be included in the method 700. As an example, the method 700 may further include receiving the digital output at the external processing system where the external processing system includes an NSP, such as the NSP 218A of FIG. 2A. In this and other embodiments, the method 700 may further include performing, by the NSP, signal processing on the digital output. In particular, the NSP may perform one or more of the signal processing functions described above with respect to the NSP 218A of FIG. 2A.

As another example, the method 700 may further include receiving the digital output at the external processing system where the external processing system includes a DAC, a front-end amplifier, and an NSP, such as the DAC 224, front-end amplifier 226 and NSP 218B of FIG. 2B. In this and other embodiments, the method 700 may further include converting the digital output to an analog signal for further conditioning/processing by an analog front-end amplifier and analog NSP.

FIG. 7B is a flowchart of an example method 720A of conditioning collected analog neural signals that may correspond to the act 720 of FIG. 7A. The method 720A of FIG. 7B is implemented in some embodiments by an implantable electronics package, such as the implantable electronics packages 210, 304, 400A, 400B, 500 of FIGS. 2A-4B, including an amplifier circuit, such as the amplifier circuits 502, 600 of FIGS. 5-6, as well as ADC circuits and a controller circuit, such as the ADC circuits 504, 506 and controller circuit 508 of FIG. 5.

The method 720A begins in some embodiments by amplifying 721 analog neural signals collected by a plurality of electrodes from a test subject. The act 721 of amplifying the collected analog neural signals is performed in some embodiments by pre-amplifiers of an amplifier circuit included in an implantable electronics package, such as the pre-amplifiers 604-606 of FIG. 6.

At act 722, the amplified analog neural signals are filtered within the test subject using a bandpass filter. The act 722 of filtering the amplified analog neural signals is performed in some embodiments by filters of an amplifier circuit included in an implantable electronics package, such as the filters 607-609 of FIG. 6.

At act 723, the filtered analog neural signals are multiplexed within the test subject to generate a plurality of multiplexed analog neural signals, where a number of the multiplexed analog neural signals is less than a number of the collected analog neural signals. The act 723 of multiplexing the filtered analog neural signals is performed in some embodiments by multiplexers of an amplifier circuit included in an implantable electronics package, such as the multiplexers 610-612 of FIG. 6.

At act 724, the multiplexed analog neural signals are digitized within the test subject to generate a corresponding number of digital neural signals. The act 724 of digitizing the multiplexed analog neural signals is performed in some embodiments by ADC circuits included in an implantable electronics package, such as the ADC circuits 504, 506 of FIG. 5.

At act 725, the digital neural signals are packetized within the test subject for inclusion in a single digital output. The act 725 of packetizing the digital neural signals is performed in some embodiments by a controller circuit included in an implantable electronics package, such as the controller circuit 508 of FIG. 5.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An implantable neural signal acquisition apparatus comprising:
a plurality of electrodes configured to be subcutaneously implanted within neural tissue of a test subject and to collect analog neural signals from the test subject;
an implantable electronics package configured to be subcutaneously implanted within the test subject and to convert the analog neural signals to digital output; and
a wire bundle coupled between the electrode array and the implantable electronics package and configured to convey the analog neural signals from the plurality of electrodes to the implantable electronics package.

2. The implantable neural signal acquisition apparatus of claim 1, wherein the wire bundle is a first wire bundle, further comprising a second wire bundle coupled to an output of the implantable electronics package and configured to convey the digital output external to the test subject.

3. The implantable neural signal acquisition apparatus of claim 2, wherein the second wire bundle comprises a single pigtail having a plurality of ring contacts on a first end opposite a second end coupled to the implantable electronics package.

4. The implantable neural signal acquisition apparatus of claim 1, wherein the implantable electronics package comprises a printed circuit board assembly and a bio-compatible housing.

5. The implantable neural signal acquisition apparatus of claim 4, wherein the bio-compatible housing comprises a polymer layer coating the printed circuit board assembly and a bio-compatible silicone layer coating the polymer layer.

6. The implantable neural signal acquisition apparatus of claim 5, wherein the polymer layer comprises a p-xylylene derivative.

7. The implantable neural signal acquisition apparatus of claim 4, wherein the bio-compatible housing comprises titanium.

8. The implantable neural signal acquisition apparatus of claim 4, wherein the printed circuit board assembly comprises:
an amplifier circuit configured to receive the analog neural signals;
a plurality of analog-to-digital converters, each coupled to a respective output of the amplifier circuit; and
a controller coupled to an output of each of the plurality of analog-to-digital converters.

9. The implantable neural signal acquisition apparatus of claim 1, wherein a length of the wire bundle is between about 1.5 centimeters and about 30 centimeters.

10. The implantable neural signal acquisition apparatus of claim 1, wherein a length of the wire bundle is between about 5 centimeters and about 24 centimeters.

11. The implantable neural signal acquisition apparatus of claim 1, wherein the implantable electronics package is further configured to multiplex the analog neural signals such that a number of digital signals in the digital output is less than a number of analog neural signals received from the electrode array.

12. A neuralphysiological data acquisition system comprising:
an implantable neural signal acquisition apparatus including:
an electrode array configured to be implanted subcutaneously within neural tissue of a test subject;
a wire bundle coupled to the electrode array and configured to be implanted subcutaneously within the test subject; and
an implantable electronics package coupled to the wire bundle and configured to be implanted subcutaneously within the test subject, the implantable electronics package further configured to convert the analog neural signals to digital output; and
an external neural signal processor communicatively coupled to the implantable neural signal acquisition apparatus.

13. The neuralphysiological data acquisition system of claim 12, further comprising a computer communicatively coupled to the external neural signal processor and configured to receive an output of the external neural signal processor.

14. The neuralphysiological data acquisition system of claim 12, further comprising an optical channel configured to communicatively couple the implantable neural signal acquisition apparatus to the external neural signal processor.

15. The neuralphysiological data acquisition system of claim 12, further comprising:
a front-end amplifier coupled between the implantable neural signal acquisition apparatus and the external neural signal processor; and
a digital-to-analog converter coupled between the implantable neural signal acquisition apparatus and the front-end amplifier.

16. A method of collecting and conditioning neural signals comprising:
collecting analog neural signals from neural tissue of a test subject;
conditioning the collected analog neural signals within the test subject to generate a single digital output representing the collected analog neural signals; and
transmitting the single digital output outside of the test subject to an external processing system.

17. The method of claim 16, wherein conditioning the collected analog neural signals within the test subject to generate a digital output comprises:
amplifying the collected analog neural signals within the test subject;
filtering the amplified analog neural signals within the test subject using a bandpass filter;
multiplexing the filtered analog neural signals within the test subject to generate a first number of multiplexed analog neural signals that is less than a second number of amplified analog neural signals;
digitizing the multiplexed analog neural signals within the test subject to generate a corresponding number of digital neural signals; and
packetizing the digital neural signals within the test subject for inclusion in the single digital output.

18. The method of claim 16, wherein transmitting the digital output to the external processing system includes converting the digital output to an optical signal and transmitting the optical signal to the external processing system via an optical transmission channel.

19. The method of claim 16, further comprising:
receiving the digital output at the external processing system including a neural signal processor; and
performing, by the neural signal processor, one or more signal processing functions on the digital output.

20. The method of claim 19, wherein performing one or more signal processing functions on the digital output comprises at least one of:
digitally filtering the digital output based on one or more specific filter criteria;
detecting neuron action potentials embedded in the digital output;
using a particular electrode of a subcutaneously implanted electrode array used to collect the analog neural signals as a reference electrode; or using bipolar differential reporting.
